(19) United States
(12) Patent Application Publication
(10) Pub. No.: US 2023/0395188 A1
(43) Pub. Date: Dec. 7, 2023

(54) SYSTEMS AND METHODS FOR RECOMMENDING ACTIVITIES OR ACTIONS TO ENHANCE GENETIC EXPRESSION LEADING TO PHENOTYPICAL TRAITS

(71) Applicant: J Ann Dunn, Cumming, GA (US)

(72) Inventor: J Ann Dunn, Cumming, GA (US)

(21) Appl. No.: 17/834,844

(22) Filed: Jun. 7, 2022

(51) Int. Cl. G16B 20/00; G16B 50/30; G16H 10/60; G16H 20/60; G16H 50/30

(52) U.S. Cl. CPC G16B 20/00; G16B 50/30; G16H 10/60; G16H 20/60; G16H 50/30

(57) ABSTRACT

Correlations stored in databases and an individual's DNA test results are used to recommend an activity to the individual to enhance expression of a gene that influences a phenotypical health trait. The databases include a pathway database, and an activity database. The pathway database stores gene correlations that indicate correlations between genes and biochemical pathways. The biochemical pathways influence the individual's phenotypical health traits. The activity database stores activity correlations that indicate correlations between human activities and biochemical pathway effects. The DNA test result includes gene indicators indicating the individual's genetic makeup. The pathway database and the gene indicators are used to identify the individual's influenced biochemical pathways. The activity database is used to identify the activity because the activity affects at least one of the influenced biochemical pathways. The activity is recommended to the individual.

### TECHNICAL FIELD

The embodiments herein relate to databases that include correlations between genes and biochemical pathways, databases that include correlations between activities and biochemical pathways, genetic testing, and inferencing to identify activities that may affect the development of an individual's likely phenotypical traits.

### BACKGROUND

The observable characteristics of an individual are that individual's phenotypical traits. Those phenotypical traits are often the result of the biochemical pathways that are active within that individual. Biochemical pathways can be activated through the expression of an individual's genes. However, two individuals with the same genes will not necessarily exhibit the same phenotypical traits because different genes may be expressed based on each individual's activities such as dietary activities, physical activities, environmental exposure, etc. In the past, the presence or state of a specific gene has been used to diagnose a specific phenotypical trait or to infer a risk of a specific phenotypical trait. For example, the genetics of a lactose intolerant person can inhibit the biochemical pathway for processing lactose from being active (a required enzyme is not produced). In such a case, an individual can present with symptoms of lactose intolerance and, based on a genetic test, can be diagnosed as incapable of producing a required enzyme because the individual's genetics have been correlated with lactose intolerance.

### BRIEF SUMMARY OF SOME EXAMPLES

The following presents a summary of one or more aspects of the present disclosure, in order to provide a basic understanding of such aspects. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure in a form as a prelude to the more detailed description that is presented later.

One aspect of the subject matter described in this disclosure can be implemented in a method. The method can include maintaining a pathway database indicating a plurality of gene correlations between a plurality of genes and a plurality of biochemical pathways that influence a plurality of phenotypical health traits, maintaining an activity database indicating a plurality of activity correlations between a plurality of human activities and a plurality of biochemical pathway effects, receiving a DNA test result that includes a plurality of gene indicators that indicate a genetic makeup of an individual, using the pathway database and the gene indicators to identify a plurality of influenced biochemical pathways of the individual, using the activity database to identify an activity that affects at least one of the influenced biochemical pathways, and recommending the activity to the individual to enhance expression of a gene that influences a phenotypical health trait that is one of the phenotypical health traits.

Another aspect of the subject matter described in this disclosure can be implemented by a system. The system can include a pathway database that indicates a plurality of gene correlations between a plurality of genes and a plurality of biochemical pathways that influence a plurality of phenotypical health traits, an activity database indicating a plurality of activity correlations between a plurality of human activities and a plurality of biochemical pathway effects, a memory, and a processor communicatively coupled to the memory. The processor can be configured to receive a DNA test result that includes a plurality of gene indicators that indicate a genetic makeup an individual, use the pathway database and the gene indicators to identify a plurality of influenced biochemical pathways of the individual, use the activity database to identify an activity that affects at least one of the influenced biochemical pathways, and produce a recommendation for the individual to perform the activity to enhance expression of a gene that influences a phenotypical health trait that is one of the phenotypical health traits.

Yet another aspect of the subject matter described in this disclosure can be implemented by a system. The system can include a means for identifying a plurality of influenced biochemical pathways of an individual based on a means for indicating a genetic makeup of the individual, a means for identifying an activity that affects at least one of the influenced biochemical pathways, and a means for recommending the activity for the individual to enhance expression of a gene that influences a phenotypical health trait that is one of the phenotypical health traits.

In some implementations of the methods and devices, the method further includes identifying an expressed phenotypical health trait of the individual, wherein the activity is recommended to enhance the expressed phenotypical health trait. In some implementations of the methods and devices, the expressed phenotypical health trait is identified based on an online questionnaire. In some implementations of the methods and devices, the expressed phenotypical health trait is identified using a telepresence interview.

In some implementations of the methods and devices, the activity is eating a food that enhances the phenotypical health trait via the at least one of the influenced biochemical pathways. In some implementations of the methods and devices, the activity is avoiding a food that promotes the phenotypical health trait via the at least one of the influenced biochemical pathways. In some implementations of the methods and devices, the activity is taking a dietary supplement that suppresses the phenotypical health trait via the at least one of the influenced biochemical pathways. In some implementations of the methods and devices, the gene is one of a plurality of genes that govern a biochemical pathway associated with the phenotypical health trait. In some implementations of the methods and devices, a second gene influences the phenotypical health trait and influences a second phenotypical health trait, wherein the activity does not enhance expression of the second gene. In some implementations of the methods and devices, the method further includes identifying a second gene that influences the phenotypical health trait, and determining that the second gene also influences a second phenotypical health trait, wherein the activity is selected instead of a second activity that enhances expression of the gene and the second gene.

In some implementations of the methods and devices, an expressed phenotypical health trait of the individual is determined using an online questionnaire. In some implementations of the methods and devices, the activity is recommended to enhance the expressed phenotypical health trait. In some implementations of the methods and devices, the phenotypical health trait is enhanced by the activity via the at least one of the influenced biochemical pathways. In some implementations of the methods and devices, a second gene is identified that influences the phenotypical health trait and influences a second phenotypical health trait, and the activity does not enhance expression of the second gene. In some implementations of the methods and devices, a second gene influences the phenotypical health trait, the second gene also influences a second phenotypical health trait, and the activity is selected instead of a second activity because expression of the second gene is enhanced by the second activity.

In some implementations of the methods and devices, the system further includes a means for identifying an expressed phenotypical health trait of the individual, wherein the activity is recommended to enhance the expressed phenotypical health trait. In some implementations of the methods and devices, the gene is one of a plurality of genes that govern a biochemical pathway associated with the phenotypical health trait. In some implementations of the methods and devices, the system further includes a means for identifying a second gene that influences the phenotypical health trait, a means for determining that the second gene also influences a second phenotypical health trait, and a means for not recommending a second activity wherein expression of the gene and of the second gene is enhanced by the second activity.

These and other aspects will become more fully understood upon a review of the detailed description, which follows. Other aspects, features, and embodiments will become apparent to those of ordinary skill in the art, upon reviewing the following description of specific, exemplary embodiments in conjunction with the accompanying figures. While features may be discussed relative to certain embodiments and figures below, all embodiments can include one or more of the advantageous features discussed herein. In other words, while one or more embodiments may be discussed as having certain advantageous features, one or more of such features may also be used in accordance with the various embodiments discussed herein. In similar fashion, while exemplary embodiments may be discussed below as device, system, or method embodiments such exemplary embodiments can be implemented in various devices, systems, and methods.

### BRIEF DESCRIPTION OF THE DRAWINGS

FIG. 1 is a high-level functional diagram illustrating recommending an activity to an individual based on gene indicators and identified expressed phenotypical traits according to some aspects.

FIG. 2 is a high-level functional diagram illustrating a relationship between an individual's genetic makeup, influenced biochemical pathways, and expressed phenotypical traits according to some aspects.

FIG. 3 is a high-level block diagram illustrating a computer supporting systems and methods for recommending activities or actions to enhance genetic expression leading to positive phenotypical traits according to some aspects.

FIG. 4 is a high-level block diagram illustrating a software system, according to some aspects.

FIG. 5 is a high-level conceptual diagram illustrating a pathway database according to some aspects.

FIG. 6 is a high-level conceptual diagram illustrating a health trait database according to some aspects.

FIG. 7 is a high-level conceptual diagram illustrating an activity database according to some aspects.

FIG. 8 is a high-level flow diagram illustrating a process that identifies influenced biochemical pathways according to some aspects.

FIG. 9 is a high-level flow diagram illustrating a process that identifies candidate biochemical pathways according to some aspects.

FIG. 10 is a high-level flow diagram illustrating a process that ensures that the influenced biochemical pathways are consistent with the expressed phenotypical health traits according to some aspects.

FIG. 11 is a high-level flow diagram illustrating a process that identifies candidate activities according to some aspects.

FIG. 12 is a high-level flow diagram illustrating a process that determines an amount of side effects for an activity according to some aspects.

FIG. 13 is a high-level flow diagram illustrating a process that selects an activity from the candidate activities according to some aspects.

FIG. 14 is a high-level flow diagram illustrating a process that recommends an activity according to some aspects.

FIG. 15 is a high-level flow diagram illustrating a method for recommending activities or actions to suppress genetic expression leading to phenotypical traits according to some aspects.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment”, “an embodiment”, or similar language means that a particular feature, structure, or characteristic described in connection with the indicated embodiment is included in at least one embodiment of the present invention. Thus, the phrases “in one embodiment”, “in an embodiment”, and similar language throughout this specification may, but do not necessarily, all refer to the same embodiment.

In the past, genetic testing has been used as a diagnostic tool and for inferring risk. As a diagnostic tool, the presence or lack of a specific gene suggests that a person's genetics are the reason the person exhibits a particular symptom. For inferring risk, the presence or lack of a specific gene suggests that a person is likely or unlikely to suffer a disorder at some future time. A more holistic and nuanced approach can use genetic data, knowledge of biophysical pathways, and the effects of certain activities on gene expression to improve an individual's overall health. There are numerous biochemical pathways in the body. A person's genetics, environment, and activities can influence the activation or inhibition of their biological pathways. Furthermore, the biochemical pathways can influence the expression of genes. For example, a biochemical pathway can include DNA methylation. DNA methylation, often simply called methylation, is a biochemical process that adds methyl groups to DNA molecules. Methylation can change the activity of a DNA segment. In many cases, methylation represses gene transcription. More succinctly, methylation is one process by which a person's activities can affect the expression of their genes.

The expression of a gene can influence a phenotypical trait via the gene's effect on a biochemical pathway. For example, height is a phenotypical trait and there are many genes that can influence height via their effects on biochemical pathways that encourage growth. Certain activities (e.g., dietary intakes, rest, exercise, etc.) can affect the expression of the genes that influence height. It is therefore possible to, in essence, engineer an individual's height, with reason, by suppressing or not suppressing specific genes that influence height.

While engineering height is an interesting possibility, it is even more useful to engineer the expression of phenotypical health traits (PHTs) Phenotypical health traits can relate to physical or mental health. For example, depression, fatigue, migraine, and soreness are phenotypical health traits. Similarly, the lack of depression, lack of fatigue, lack of migraine, and lack of soreness are phenotypical health traits. PHTs can be encouraged or discouraged through specific activities (e.g., dietary intakes, rest, exercise, etc.). The activities affect the activation or suppression of genes. The genes influence the PHTs via biochemical pathways.

One of the difficulties is determining which biochemical pathways to influence in order to encourage or discourage a phenotypical trait because each phenotypical trait may be affected by a number of biochemical pathways and because each biochemical pathway may affect a number of phenotypical traits. Returning to the height example, enhancing a specific biochemical pathway may lead to increased height and also to depression. As such, the biochemical pathways to enhance must be chosen with care. Another difficulty is choosing which genes to suppress or activate because each gene may influence a number of biochemical pathways. As such, the genes to activate or suppress must be chosen with care.

Some phenotypical traits are considered positive phenotypical traits and others are considered negative phenotypical traits. Bone density is considered to be positive phenotypical traits. Anxiety is considered to be a negative phenotypical trait. Vitamin D is believed to stimulate the absorption of calcium and may thereby enhance bone density. Note that slowing the loss of bone density is considered an enhancement of bone density. The VDR gene provides instructions for making a protein called vitamin D receptor (VDR), which allows the body to respond to vitamin D. The VDR gene also affects adrenaline, a hormone excreted by the adrenal glands. Anxiety is very highly linked to the presence of adrenaline. If we enhance the expression of the VDR gene it will increase the production of adrenalin. A different gene, the COMT gene, also affects adrenaline. The COMT gene provides instructions for making an enzyme called catechol-O-methyltransferase (COMT). The COMT enzyme breaks down adrenaline. Enhancing the expression of the VDR gene can increase the production of adrenalin and cause a problem downstream if another gene (e.g., the COMT gene) is not functioning well or not enhanced. It can lead to increased anxiety.

Enhancing expression of the VDR gene can lead to the suppression of negative phenotypical traits associated with vitamin D deficiency (e.g., rickets). Such suppression may be seen as enhancements of positive phenotypical traits associated with vitamin D uptake, such as an increase (or less of a decrease) in bone density. Enhancing expression of the VDR gene may also lead to the enhancement of anxiety. Enhancing expression of the COMT gene can lead to the suppression anxiety.

Eating/avoiding certain foods, taking certain dietary supplements, laying in the sun (for vitamin D), and other activities can enhance or suppress biochemical pathways. A patient can receive a recommendation to avoid certain foods to thereby slow down the function of an enzyme and cause further adverse phenotype expression (e.g., cause an adverse health trait to be expressed). In other cases, a dietary supplement may be recommended that has an effect on a biochemical pathway that enhances the expression of a positive health trait. the recommendation can be for a dietary change that would help upregulate the function of an enzyme and enhance its activity. An example of such an enzyme is lactase, an enzyme that aids in the digestion of lactose. Taking the cofactor or eating certain foods it can make up for the inherited genetic variant that leads to a low functioning enzyme and subsequent lactose intolerance. As such, taking the cofactor or coenzyme (vitamin or mineral) may increase the activity of the enzyme and increase the patient's tolerance for lactose or their phenotypic expression of lactose intolerance.

Current and on-going research is mapping out the body's biochemical pathways and how each pathway affects phenotypical traits. Many of those phenotypical traits are phenotypical health traits (PHTs). Phenotypical health traits can relate to physical or mental health. For example, depression, fatigue, migraine, arthritis, and soreness are phenotypical health traits. Similarly, the lack of depression, lack of fatigue, lack of migraine, and lack of soreness are phenotypical health traits. The research often indicates correlations between biochemical pathways and PHTs. As such a health trait database can indicate the correlations between the activity of specific biochemical pathways and specific PHTs.

There is also current and on-going research that is mapping the effects of specific genes on specific biochemical pathways. The research often correlates the activation or suppression of specific genes and the action of biochemical pathways. As such a pathway database can indicate the correlations between the activity of specific biochemical pathways and the suppression or activation of certain genes. For example, studies of DNA methylation can indicate the correlation between the activity of specific biochemical pathways and the suppression of certain gene sequences via methylation. A methylation focused pathway database can indicate the correlations between the activity of specific biochemical pathways and the suppression of certain genes.

Yet more current and on-going research is mapping the effects of specific activities on specific biochemical pathways. More specifically, the influences of activities on biochemical pathways are being charted. As such an activity database can indicate the correlations between activities/actions and specific biochemical pathways.

A pathway database, a health trait database and an activity database can be produced by reviewing research publications and entering their findings into the appropriate database. The databases can be refined, updated and enhanced as further research is conducted and the results published. Database software running on a computer can be used to maintain the databases such that they can be queried, amended, and kept available.

A genetic test can indicate the genetic makeup of an individual. The genetic test can be used to predict which of the individual's biochemical pathways are likely to be suppressed or active. In turn, the biochemical pathways can predict the PHTs the individual has or may have in the future. Some of those PHTs may be desirable, others may be undesirable. Activities can be suggested to enhance desirable PHTs and to discourage undesirable PHTs.

A further aspect is that the individual can be evaluated to identify PHTs that are expressed or that are latent. Expressed PHTs can be observed or inferred. For example, an in-person diagnosis of depression or chronic fatigue can indicate an observed PHT. A PHT may be inferred via an indirect means such as a questionnaire or interview. Such questionnaires/interviews are used in the physical health and mental health fields to provide direction for further evaluation such as in-person or remote (e.g., telemedicine) examinations performed by clinicians. An in-person interview/examination occurs when the interviewer/examiner and the interviewee/examinee are in the same place and communicating directly. A telepresence interview/examination occurs when the interviewer/examiner and the interviewee/examinee are not in the same place and must communicate via telephone, video chat, or some other remote communications means. An interview consists of a series of questions and answers. An examination may be the same as an interview but may also be, or include, observation of physical characteristics. A questionnaire can be a textual list of questions to which a person provides written or recorded answers. Online questionnaires are commonly provided through web sites that also collect individuals' answers to the questions. Questionnaires/interviews can be analyzed to suggest PHTs that are to be given special attention when evaluating genetic and biochemical pathway data. Questionnaires are often produced such that yes/no or multiple-choice answers can be automatically analyzed to thereby infer aspects, situations, or conditions of the person responding to the questionnaire. Such questionnaires may also be automatically analyzed to infer PHTs. Inferring a likely PHT can be valuable because the genetic and biochemical data may indicate that the individual is also at risk of that PHT. As such, a minor lifestyle or dietary change may inhibit the full expression of the PHT.

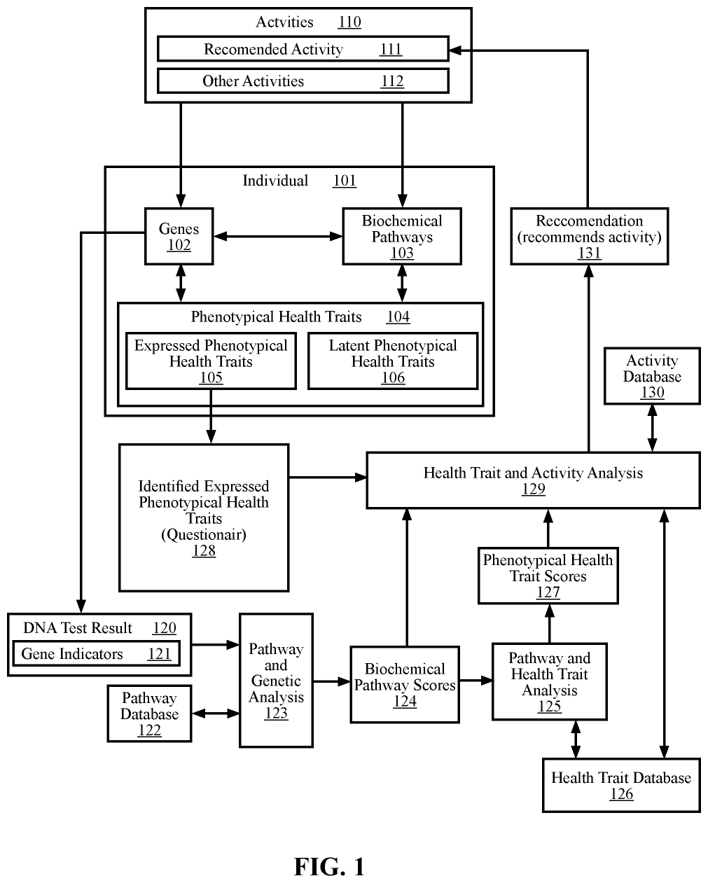

FIG. 1 is a high-level functional diagram illustrating recommending an activity to an individual based on gene indicators and identified expressed phenotypical traits according to some aspects. An individual 101 has genes 102 and biochemical pathways 103. The genes 102 affect the biochemical pathways 103 and the biochemical pathways 103 affect the genes 102. The person performs various activities 110 that affect the genes 102 and the biochemical pathways 103. In particular, the activities 110 can affect biochemical pathways 103 that affect the genes 102. The individual also has phenotypical health traits (PHTs) 104 that include expressed PHTs 105 and latent PHTs 106. The expressed PHTs 105 are PHTs 104 that may be observed. The latent PHTs 106 are PHTs 104 that the individual is at risk of having, but are not observable and are thereby far below the level of requiring full medical intervention (e.g., surgery, prescription drugs, etc.). A DNA test result 120 can be obtained via genetic testing of the individual. Such testing is often performed by professional genetic testing laboratories. The DNA test result 120 can include a number of gene indicators 121 that indicate the individual's genetics. Pathway and genetic analysis 123 can use the DNA test results 120 and a pathways database 122 to calculate biochemical pathway scores 124. The biochemical pathway scores 124 can indicate influenced biochemical pathways. The influenced biochemical pathways can be the biochemical pathways having the highest biochemical pathway scores 124 and are thereby also the biochemical pathways that are most likely to be influenced by the individual's genetics. Pathway and health trait analysis 125 can use the biochemical pathway scores 124 and a health trait database 126 to calculate phenotypical health trait scores 127.

Certain of the individual's expressed PHTs 105 are identified PHTs 128. For example, certain PHTs may be automatically identified from a questionnaire that has been filled out by the individual. Health trait and activity analysis 129 can use the identified PHTs 128, the biochemical pathway scores 124, the phenotypical health trait scores 127, and an activity database 130 to identify a recommended activity 111. Health trait and activity analysis 129 can produce a recommendation 131 of the recommended activity 111. The individual 101 can receive the recommendation 131 and thereby be informed of the recommended activity 111. As such, the activities 110 performed by the individual 101 can include the recommended activity 111 as well as other activities 112.

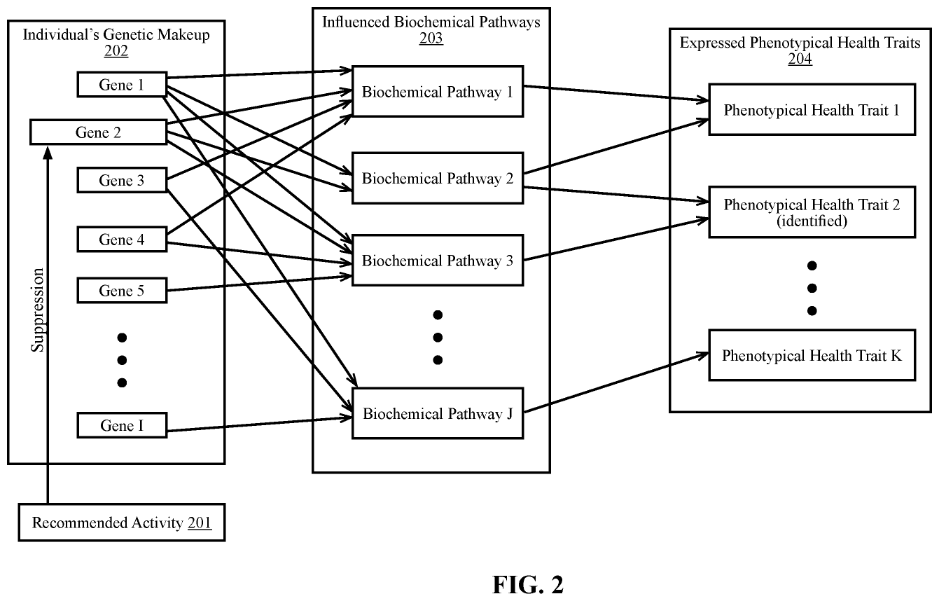

FIG. 2 is a high-level functional diagram illustrating a relationship between an individual's genetic makeup 202, influenced biochemical pathways 203, and expressed phenotypical traits 204 according to some aspects. The individual's genetic makeup 202 can include genes such as gene 1, gene 2, gene 3, gene 4, gene 5, gene I, etc. The genes affect the biochemical pathways within the individual's body. The illustration indicates that biochemical pathway 1 is affected by gene 1, gene 3, and gene 4. The illustration also indicates that biochemical pathway 2 is affected by gene 1, and gene 2. The illustration additionally indicates that biochemical pathway 3 is affected by gene 1, gene 2, gene 4, and gene 5. The illustration further indicates that biochemical pathway J is affected by gene 1, gene 3, and gene I. The influenced biochemical pathways 203 can lead to expressed phenotypical health traits 204. The illustration indicates that phenotypical health trait 1 is affected by biochemical pathway 1, and by biochemical pathway 2. The illustration also indicates that phenotypical health trait 2 is affected by biochemical pathway 2, and by biochemical pathway 3. The illustration further indicates that phenotypical health trait K is affected by biochemical pathway J. Here, phenotypical health trait 2 has been identified and an activity is to be recommended to reduce PHT 2. It can be seen that enhancing gene 1 would affect phenotypical health trait 1, phenotypical health trait 2, phenotypical health trait K, and possibly others. As such, it may be best to leave gene 1 alone. It can also be seen that enhancing gene I would not affect phenotypical health trait 2 and should be left alone. However, it can be seen from the figure that gene 2 affects phenotypical health trait 2 via biochemical pathway 2 and biochemical pathway 3. It is therefore determined that gene 2 should be enhanced. Gene 2 may be enhanced through diet and supplement recommendations. It is understood that such enhancement occurs when the recommendations are followed. Recommended activity 201 is known to suppress gene 2. It can therefore be suggested to the individual that they perform recommended activity 201.

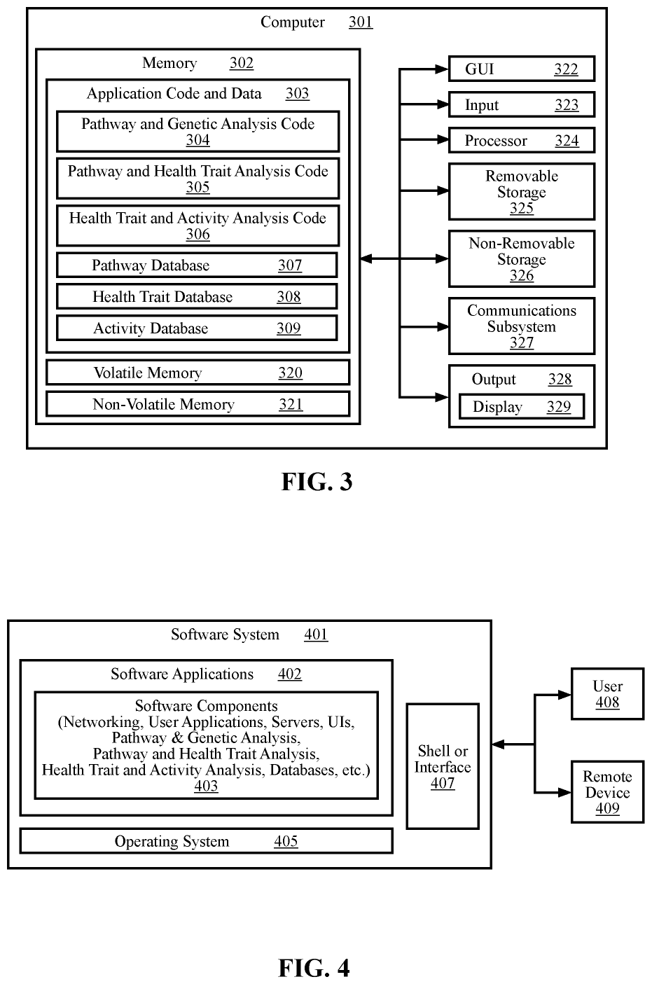

FIG. 3 is a high-level block diagram illustrating a computer 301 supporting systems and methods for recommending activities or actions to enhance genetic expression leading to positive phenotypical traits according to some aspects. A computing device in the form of a computer 301 configured to interface with controllers, peripheral devices, and other elements disclosed herein may include one or more processors 324, memory 302, removable storage 325, and non-removable storage 326. Memory 302 may include volatile memory 320 and non-volatile memory 321. Computer 301 may include or have access to a computing environment that includes a variety of transitory and non-transitory computer-readable media such as volatile memory 320 and non-volatile memory 321, removable storage 325 and non-removable storage 326. Computer storage includes, for example, random access memory (RAM), read only memory (ROM), erasable programmable read-only memory (EPROM) and electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage, or other magnetic storage devices, or any other medium capable of storing computer-readable instructions and data.

Computer 301 may include, or have access to, a computing environment that includes input 323, output 328, and a communications subsystem 327. The computer 301 may operate in a networked environment using a communications subsystem 327 to connect to one or more remote computers, remote sensors and/or controllers, detection devices, hand-held devices, multi-function devices, speakers, mobile devices, tablet devices, mobile phones, Smartphone, or other such devices. The remote computer may also be a personal computer (PC), server, router, network PC, RFID enabled device, a peer device or other common network node, or the like. The communication connection may include a LAN, a WAN, Bluetooth connection, or other networks.

Output 328 is most commonly provided as a computer monitor or display 329, but may include any output device. Output 328 and/or input 323 may include a data collection apparatus associated with computer 301. In addition, input 323, which commonly includes a computer keyboard and/or pointing device such as a computer mouse, computer trackpad, touch screen, or the like, allows a user to select and instruct computer 301. A user interface can be provided using output 328 and input 323. Output 328 may include a display 329 for displaying data and information for a user, or for interactively displaying a GUI (graphical user interface) 322. A GUI 322 is typically responsive to user inputs entered through input 323 and typically displays images and data on display 329.

Note that the term “GUI” generally refers to a type of environment that represents programs, files, options, and so forth by means of graphically displayed icons, menus, and dialog boxes on a computer monitor screen or smart phone screen. A user can interact with the GUI to select and activate such options by directly touching the screen and/or pointing and clicking with a user input device 323 such as, for example, a pointing device such as a mouse, and/or with a keyboard. A particular item can function in the same manner to the user in all applications because the GUI provides standard software routines (e.g., the application code and data 303 can include program code in executable instructions, including such software routines) to handle these elements and report the user's actions.

Computer-readable instructions, for example, program code in application code and data 303, can include or be representative of software routines, software subroutines, software objects, etc. described herein, are stored on a computer-readable medium and are executable by the processor device (also called a processing unit) 324 of computer 301. The application code and data 303 can include computer code such as pathway and genetic analysis code 304, pathway and health trait analysis code 305, health trait and activity analysis code 306, a pathway database 307, a health trait database 308, and an activity database 309. A hard drive, CD-ROM, RAM, Flash Memory, and a USB drive are just some examples of articles including a computer-readable medium.

FIG. 4 is a high-level block diagram illustrating a software system 401, according to some aspects. Software applications 402, may be stored in memory 302, on removable storage 325, or on non-removable storage 326, and generally includes and/or is associated with a kernel or operating system 405 and a shell or interface 407. One or more application programs may be “loaded” (i.e., transferred from removable storage 325 or non-removable storage 326 into the memory 302) for execution by the computer 301. An application program can include software components 403 such as software modules, software subroutines, software objects, network code, user application code, server code, UI code, pathway & genetic analysis code, pathway and health trait analysis code, health trait and activity analysis code, databases, etc. The databases can include a pathway database 307, a health trait database 308, and an activity database 309. The software system 401 can have multiple software applications each containing software components. The computer 301 can receive user commands and data through interface 407, which can include input 323, output 328, and communications connection 327 accessible by a user 408 or remote device 409. These inputs may then be acted upon by the computer 301 in accordance with instructions from operating system 405 and/or software application 402 and any software components 403 thereof.

Generally, software components 402 can include, but are not limited to, routines, subroutines, software applications, programs, objects, modules, objects (used in object-oriented programs), executable instructions, data structures, etc., that perform particular tasks or implement particular abstract data types and instructions. Moreover, those skilled in the art will appreciate that elements of the disclosed methods and systems may be practiced with other computer system configurations such as, for example, hand-held devices, mobile phones, smartphones, tablet devices, multi-processor systems, microcontrollers, printers, copiers, fax machines, multi-function devices, data networks, microprocessor-based or programmable consumer electronics, networked personal computers, minicomputers, mainframe computers, servers, medical equipment, medical devices, and the like.

Note that the terms “component,” “module” as utilized herein may refer to one of or a collection of routines and data structures that perform a particular task or implements a particular abstract data type. Applications and components may be composed of two parts: an interface, which lists the constants, data types, variables, and routines that can be accessed by other modules or routines; and an implementation, which is typically private (accessible only from within the application or component) and which includes source code that actually implements the routines in the application or component. The terms application or component may also simply refer to an application such as a computer program designed to assist in the performance of a specific task such as word processing, accounting, inventory management. Components can be built or realized as special purpose hardware components designed to equivalently assist in the performance of a task.

The interface 407 can include a graphical user interface 322 that can display results, whereupon a user 408 or remote device 409 may supply additional inputs or terminate a particular session. In some embodiments, operating system 405 and GUI 322 can be implemented in the context of a “windows” system. It can be appreciated, of course, that other types of systems are possible. For example, rather than a traditional “windows” system, other operating systems such as, for example, a real-time operating system (RTOS) more commonly employed in wireless systems may also be employed with respect to operating system 405 and interface 407. The software application 402 can include, for example, software components 403, which can include instructions for carrying out steps or logical operations such as those shown and described herein.

The description herein is presented with respect to embodiments that can be embodied in the context of, or require the use of, a data-processing system such as computer 301, in conjunction with program code in application code and data 303 in memory 302, software system 401, or computer 301. The disclosed embodiments, however, are not limited to any particular application or any particular environment. Instead, those skilled in the art will find that the system and method of the present invention may be advantageously applied to a variety of system and application software including database management systems, word processors, and the like. Moreover, the present invention may be embodied on a variety of different platforms including Windows, Macintosh, UNIX, LINUX, Android, Arduino, and the like. Therefore, the descriptions of the exemplary embodiments, which follow, are for purposes of illustration and not considered a limitation.

Computer 301 and software systems 401 can take the form of or run as virtual machines (VMs) or containers that run on physical machines. A VM or container typically supplies an operating environment, appearing to be an operating system, to program code in an application module and software applications 402 running in the VM or container. A single physical computer can run a collection of VMs and containers. In fact, an entire network data processing system including a multitude of computers, LANs and perhaps even WANs or portions thereof can all be virtualized and running within a single computer (or a few computers) running VMs or containers. Those practiced in cloud computing are practiced in the use of VMs, containers, virtualized networks, and related technologies.

FIG. 5 is a high-level conceptual diagram illustrating a pathway database 501 according to some aspects. The pathway database 501 associates gene identifiers 502 and biochemical pathway identifiers 503 with gene correlations. Comparing FIG. 5 and FIG. 2 it can be observed that the rows of the pathway database 501 correspond to the arrows from the individual's genetic makeup 202 to the influenced biochemical pathways 203. The values for the gene correlations 504 can be assigned based on the scientific and research literature reporting the effects of the genes on the biochemical pathways. Furthermore, a value for a gene correlation such as “gene correlation a,b” can indicate a correlation value for the effect of “gene a” on “biochemical pathway b” where “a” references the gene and “b” references the biochemical path. The gene correlations can be numerical values with larger numbers indicating greater correlation (e.g., the gene more strongly influences the pathway) between a gene and a biochemical pathway. The correlation values can be positive numbers. Correlation values may be positive and negative numbers. For example, the correlation coefficient is a well-known measure that is commonly used in the field of statistics. The absolute value of the correlation coefficient (always a positive number) indicates how strongly two variables are coordinated. The sign of the correlation coefficient indicates whether the variables are positively correlated or negatively correlated.

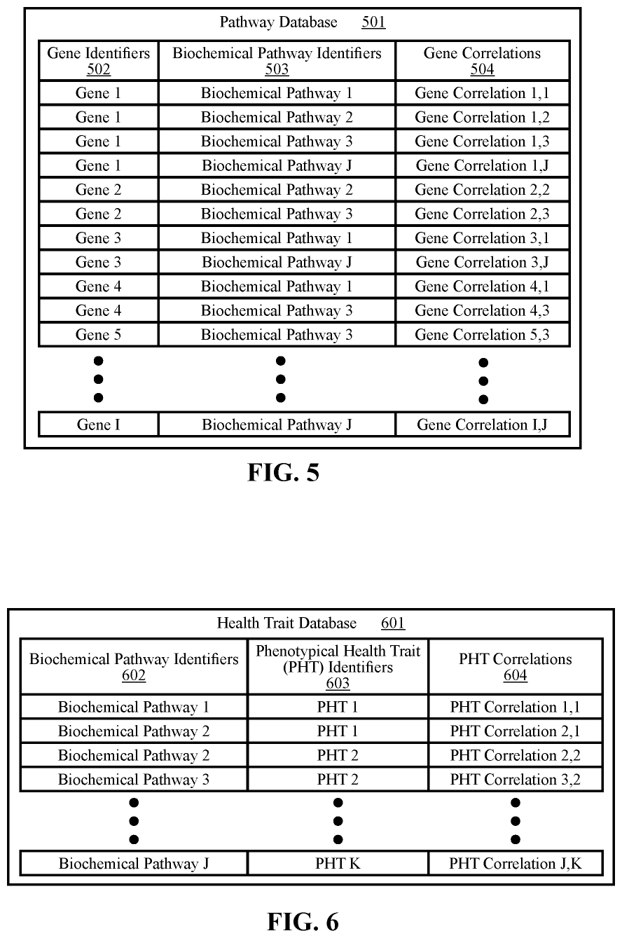

FIG. 6 is a high-level conceptual diagram illustrating a health trait database 601 according to some aspects. The health trait database 601 associates biochemical pathway identifiers 602 and PHT identifiers 603 with PHT correlations 604. Comparing FIG. 5 and FIG. 2 it can be observed that the rows of the health trait database 601 correspond to the arrows from the influenced biochemical pathways 203 to the expressed PHTs 204. The values for the PHT correlations 604 can be assigned based on the scientific and research literature reporting the effects of the biochemical pathways on the PHTs. Furthermore, a value for a PHT correlation such as “PHT correlation c,d” can indicate a correlation value for the effect of “biochemical pathway c” on “PHT d” where “c” references the biochemical path and “d” references the PHT.

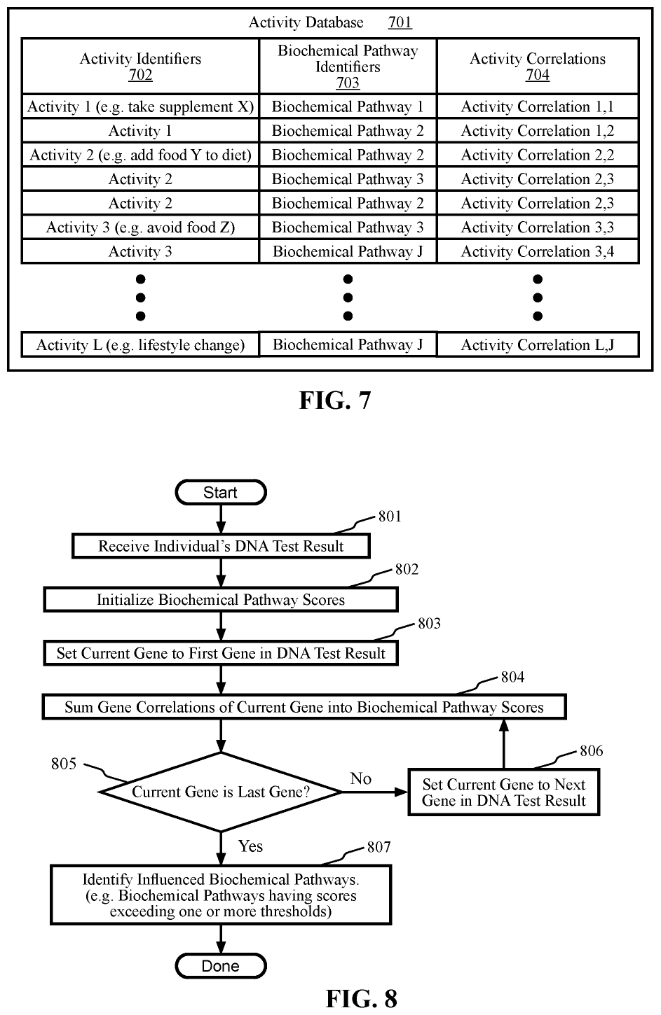

FIG. 7 is a high-level conceptual diagram illustrating an activity database 701 according to some aspects. The activity database 701 indicates correlations between human activities and biochemical pathway effects by associating activity identifiers 702 and biochemical pathway identifiers 703 with activity correlations 704. The activity correlations can indicate the strength of a biochemical pathway effect that is due to or resulting from a human activity. The activities can include dietary activities (foods, amounts eaten, etc.) lifestyle (exercising, sedentary job, smoking, drinking, outdoor activities, long/short commute, work from home, etc.), nutrients (dietary supplements, etc.), and other activities. The values for the activity correlations 704 can be assigned based on the scientific and research literature reporting the effects of various activities on the biochemical pathways. Furthermore, a value for an activity correlation such as “activity correlation e,f” can indicate a correlation value for the effect of “activity e” on “biochemical pathway f” where “f” references the biochemical path and “e” references the activity.

FIG. 8 is a high-level flow diagram illustrating a process that identifies influenced biochemical pathways according to some aspects. Here, the biochemical pathways that may be influenced by the individual's genetics may be identified. After the start, at block 801 an individual's DNA test result is received. At block 802, biochemical pathway scores are initialized. For example, the initial score for each biochemical pathway can be set to zero. At block 803, the current gene variable can be set to the first gene in the DNA test result. For example, the first gene in the DNA test result can be “gene 1” in which case current gene is set to “gene 1”. At block 804, the gene correlations associated with the current gene are summed into the biochemical pathway scores. For example, looking to the entries in pathway database associated with gene 1, gene correlation 1,1 is added to the biochemical pathway score for biochemical pathway 1, gene correlation 1,2 is added to the biochemical pathway score for biochemical pathway 2, and gene correlation 1,3 is added to the biochemical pathway score for biochemical pathway 3. At decision block 805, the process determines if current gene is the last gene in the DNA test result. If the current gene is not the last gene in the DNA test result, the process moves from decision block 805 to block 806. At block 806, current gene is set to the next gene in the DNA test result before the process loops back to block 804. If the current gene is the last gene in the DNA test result, the process moves from decision block 805 to block 807. At block 807, the influenced biochemical pathways are identified. For example, the influenced biochemical pathways can include the biochemical pathways that have a biochemical pathway score exceeding a threshold value. After block 807, the process is done.

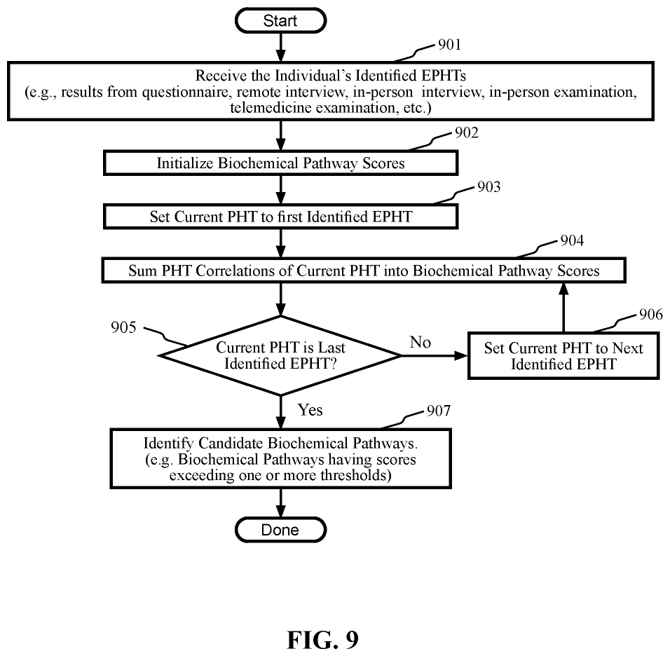

FIG. 9 is a high-level flow diagram illustrating a process that identifies candidate biochemical pathways according to some aspects. Here, the biochemical pathways that may be the root cause of the individuals EPHTs may be identified. After the start, at block 901 the process can receive the identified EPHTs of an individual. The EPHTs can be determined from a questionnaire, a remote interview, an in-person interview, an in-person examination, a telemedicine examination, etc. At block 902, the process can initialize biochemical pathway scores. At block 903, the current PHT can be set to the first identified EPHT. At block 904, the PHT correlations of the current PHT can be summed into the biochemical pathway scores. For example, looking at the entries in the health trait database, if PHT 1 is an EPHT, then PHT correlation 1,1 may be added to the biochemical pathway score for biochemical pathway 1 and PHT correlation 2,1 may be added to the biochemical pathway score for biochemical pathway 2. If PHT 2 is an EPHT, then PHT correlation 2,2 may be added to the biochemical pathway score for biochemical pathway 2 and PHT correlation 3,2 may be added to the biochemical pathway score for biochemical pathway 3. At decision block 905, the process determines if the current PHT is the last identified EPHT. If the current PHT is not the last identified EPHT, the process moves from decision block 905 to block 906. At block 906, current PHT is set to the next identified EPHT before the process loops back to block 904. If the current PHT is the last identified EPHT, the process moves from decision block 905 to block 907. At block 907, candidate biochemical pathways are identified. For example, the candidate biochemical pathways can include the biochemical pathways that have a biochemical pathway score exceeding a threshold value. After block 907, the process is done

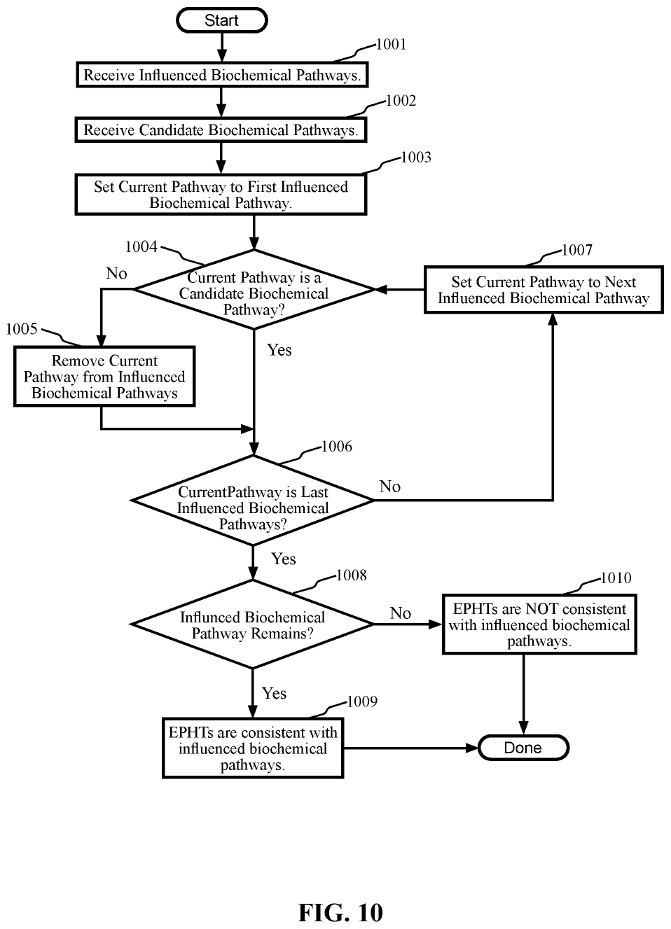

FIG. 10 is a high-level flow diagram illustrating a process that ensures that the influenced biochemical pathways are consistent with the expressed phenotypical health traits according to some aspects. After the start, at block 1001 the process can receive the influenced biochemical pathways (e.g., a list of influenced biochemical pathways). At block 1002, the process can receive the candidate biochemical pathways (e.g., a list of candidate biochemical pathways). At block 1003, the process can set the current pathway to the first influenced biochemical pathway. At decision block 1004, the process determines if the current pathway is a candidate biochemical pathway (e.g., is in the list of candidate biochemical pathways). If the current pathway is not a candidate biochemical pathway, the process moves from decision block 1004 to block 1005. At block 1005, the current pathway can be removed from the influenced biochemical pathways before the process moves to decision block 1006. If the current pathway is a candidate biochemical pathway, the process moves from decision block 1004 to decision block 1006. At decision block 1006, the process determines if the current pathway is the last influenced biochemical pathway. If the current pathway is not the last influenced biochemical pathway, the process moves from decision block 1006 to block 1007. At block 1007, the current pathway can be set to the next influenced biochemical pathway before the process loops back to decision block 1004. If the current pathway is the last influenced biochemical pathway, the process moves from decision block 1006 to decision block 1008. At decision block 1008, the process determines if any influenced biochemical pathways remain (not all were removed at block 1005). If no influenced biochemical pathways remain, the process moves from decision block 1008 to block 1010. At block 1010, the process can report that the EPHTs are not consistent with the influenced biochemical pathways before the process is done. If any influenced biochemical pathways remain, the process moves from decision block 1008 to block 1009. At block 1009, the process can report that the EPHTs are consistent with the influenced biochemical pathways before the process is done. Furthermore, the list of influenced biochemical pathways has been pruned by omitting those that are not consistent with the candidate biochemical pathways.

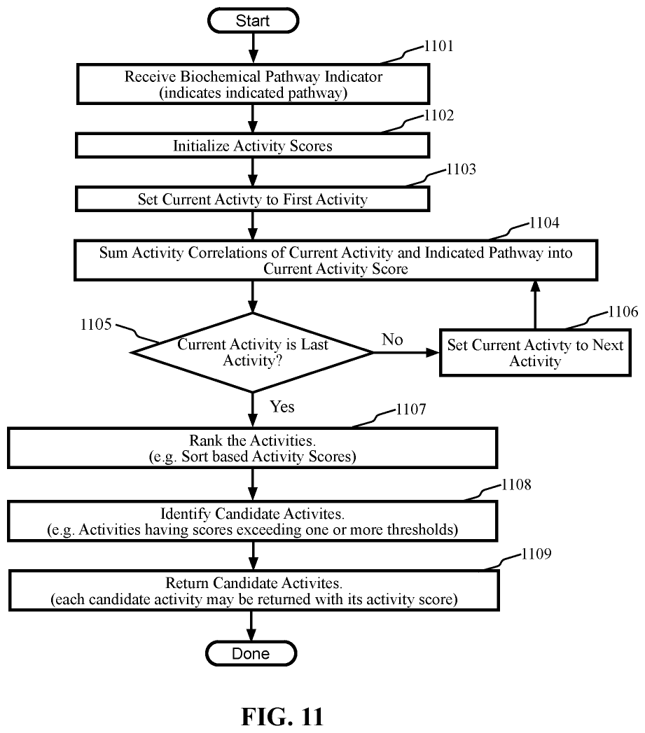

FIG. 11 is a high-level flow diagram illustrating a process that identifies candidate activities according to some aspects. Given a biochemical pathway indicator, the process can use the activity database to identify activities that affect the indicated biochemical pathway. After the start, at block 1101 the process can receive a biochemical pathway indicator. The biochemical pathway indicator indicates the indicated pathway. At block 1102, the process can initialize activity scores (e.g., each activity starts with a score of zero). At block 1103, the current activity is set to the first activity. At block 1104, the activity correlation of the current activity and the indicated pathway are summed into the current activity score. For example, if the biochemical pathway indicator indicates biochemical pathway 2 and the current activity is activity 1, then activity correlation 1,2 in the activity data base is added to the activity score for activity 1. At decision block 1105, the process determines if the current activity is the last activity. If the current activity is not the last activity, the process moves from decision block 1105 to block 1106. At block 1106, the current activity can be set to the next activity before the process loops back to block 1104. If the current activity is the last activity, the process moves from decision block 1105 to block 1107. At block 1107, the activities can be ranked by, for example, sorting them for highest scoring to lowest scoring. At block 1108, candidate activities can be identified. For example, activities having activity scores exceeding a threshold can be identified as the candidate activities. At block 1109, the candidate activities may be returned. For example, a list of candidate activities and their activity scores may be returned. Those practiced in the art of computer programming can easily adapt the process of FIG. 11 to receive multiple biochemical indicators and to loop over those multiple biochemical indicators to identify candidate activities for affecting a set of biochemical pathways. Such looping could be accomplished with an outer loop that begins just after block 1103 and ends just before block 1107.

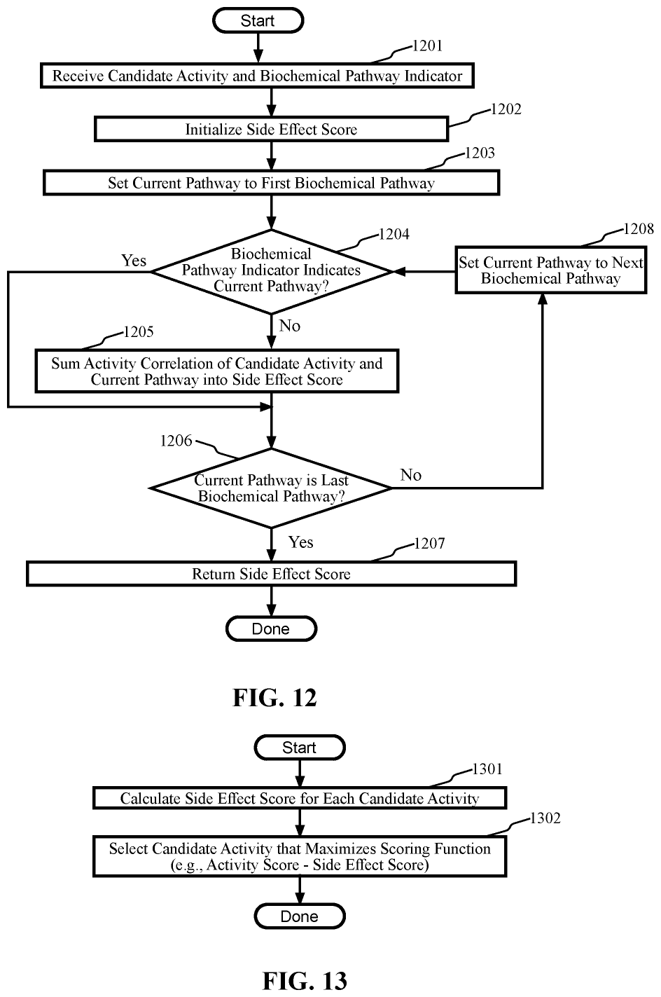

FIG. 12 is a high-level flow diagram illustrating a process that determines an amount of side effects for an activity according to some aspects. This process can loop through the activities database to add up side effects. After the start, at block 1201, the process can receive a candidate activity and a biochemical pathway indicator. The biochemical pathway indicator can indicate the pathway that is intended to be influenced. Influences on other pathways are side effects. At block 1202, a side effect score is initialized. At block 1203, the current pathway (a variable) is set to the first biochemical pathway. At decision block 1204, the process determines if the biochemical pathway indicator indicates the current pathway. If the biochemical pathway indicator indicates the current pathway, the process moves from decision block 1204 to decision block 1206. If the biochemical pathway indicator does not indicate the current pathway, the process moves from decision block 1204 to block 1205. At block 1205, the process can sum the activity correlation of the candidate activity and the current pathway into the side effect score. Such activity correlations are in the activities database. At decision block 1206, the process determines if the current pathway is the last biochemical pathway. If the current pathway is not the last biochemical pathway, the process moves from decision block 1206 to block 1208. At block 1208, the current pathway can be set to the next biochemical pathway before the process loops back to decision block 1204. If the current pathway is the last biochemical pathway, the process moves from decision block 1206 to block 1207. At block 1207, the process can return the side effect score before the process is done.

FIG. 13 is a high-level flow diagram illustrating a process that selects an activity from the candidate activities according to some aspects. After the start, at block 1301 the process can calculate side effect scores for each candidate activity. At block 1302, the process can select a candidate activity that maximizes a scoring function. For example, the scoring function can be the side effect score (calculated in FIG. 12). In another example, the scoring function can be the activity score of the candidate activity (calculated in FIG. 11) minus the side effect score. The selected candidate activity can be recommended to the individual.

The side effect score can indicate how much the activity influences a second phenotypical health trait via biochemical pathways other than the one received at block 1201. As discussed above, influenced biochemical pathways are identified based on the person's genetics (e.g., using process of FIG. 8). A process such as that of FIG. 10 can be used to prune the list of influenced pathways to include only those that are also candidate pathways. As seen in FIGS. 5-6, a person may have first gene and a second gene that are influencing a particular phenotypical health trait while the second gene also influences a second phenotypical health trait. The side effect score can indicate how strongly the second phenotypical health trait is influenced by a particular activity. The scoring function (e.g., activity score minus side effect score) can be used to select a first activity instead of a second activity such that side effects are avoided. The first activity can be selected because it enhances expression of the first gene without enhancing expression of the second gene, which gives rise to a side effect. Furthermore, correlation data such as that illustrated in FIGS. 5-6 may be used to identify that second gene.

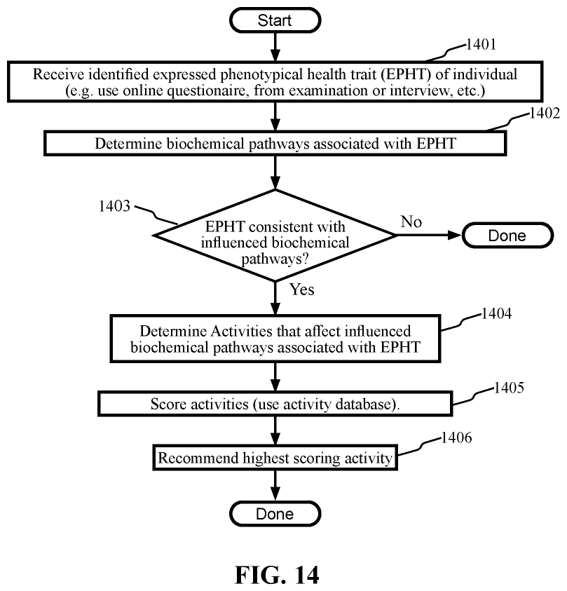

FIG. 14 is a high-level flow diagram illustrating a process that recommends an activity according to some aspects. After starting, at block 1401 the process can receive an identified EPHT of an individual. (e.g., via online questionnaire). At block 1402, the process can determine which biochemical pathways are associated with the EPHT. The health trait database may be used to determine which biochemical pathways are associated with the EPHT. At decision block 1403, the process can determine if the EPHT is consistent with the influenced biochemical pathways. The processes of FIG. 9 and FIG. 10 can be used to determine if the EPHT is consistent with the influenced biochemical pathways because the process of FIG. 9 can use the EPHT to identify candidate biochemical pathways and the process of FIG. 10 can determine if the EPHT is consistent with the candidate biochemical pathways. If the EPHT is not consistent with the influenced biochemical pathways, the process is done. If the EPHT is consistent with the influenced biochemical pathways, the process moves from decision block 1403 to block 1404. At block 1404, the process can determine which activities that affect the influenced biochemical pathways are associated with the EPHT. The process of FIG. 8 can identify influenced biochemical pathways from the individual's DNA test result. The process of FIG. 10 can identify which of the influenced biochemical pathways are associated with the EPHT. At block 1405, the process can score activities. For example, the process of FIG. 11 can return a list of activities and their activity scores at block 1109. At block 1406, the process can recommend the highest scoring activity. The highest scoring activity may be the activity with the highest activity score. Alternatively, activity with the highest score calculated using a scoring function can be the highest scoring activity. FIG. 13 illustrates a process that uses a scoring function to select a candidate activity.

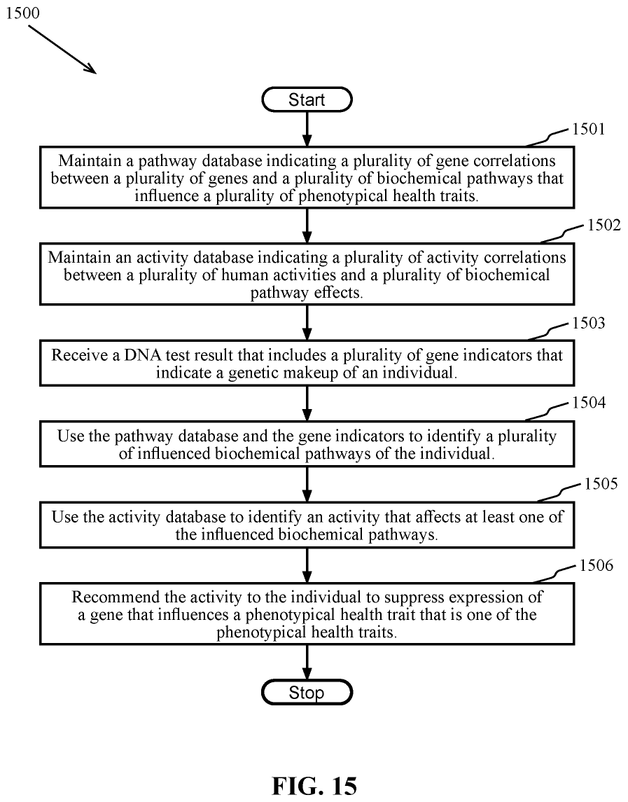

FIG. 15 is a high-level flow diagram illustrating a method for recommending activities or actions to suppress genetic expression leading to phenotypical traits according to some aspects. At block 1501, the process can maintain a pathway database indicating a plurality of gene correlations between a plurality of genes and a plurality of biochemical pathways that influence a plurality of phenotypical health traits. At block 1502, the process can maintain an activity database indicating a plurality of activity correlations between a plurality of human activities and a plurality of biochemical pathway effects. At block 1503, the process can receive a DNA test result that includes a plurality of gene indicators that indicate a genetic makeup of an individual. At block 1504, the process can use the pathway database and the gene indicators to identify a plurality of influenced biochemical pathways of the individual. At block 1505, the process can use the activity database to identify an activity that affects at least one of the influenced biochemical pathways. At block 1506, the process can recommend the activity to the individual to suppress expression of a gene that influences a phenotypical health trait that is one of the phenotypical health traits.

Although the operations of the method(s) herein are shown and described in a particular order, the order of the operations of each method may be altered so that certain operations may be performed in an inverse order or so that certain operations may be performed, at least in part, concurrently with other operations. Instructions or sub-operations of distinct operations may be implemented in an intermittent and/or alternating manner.

It should also be noted that at least some of the operations for the methods described herein may be implemented using software instructions stored on a computer usable storage medium for execution by a computer. As an example, an embodiment of a computer program product includes a computer usable storage medium to store a computer readable program.

The computer-usable or computer-readable storage medium can be an electronic, magnetic, optical, electromagnetic, infrared, or semiconductor system (or apparatus or device). Examples of non-transitory computer-usable and computer-readable storage media include a semiconductor or solid-state memory, magnetic tape, a removable computer diskette, a random-access memory (RAM), a read-only memory (ROM), a rigid magnetic disk, and an optical disk. Current examples of optical disks include a compact disk with read only memory (CD-ROM), a compact disk with read/write (CD-R/W), and a digital video disk (DVD).

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the claims as described herein.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A method comprising:
maintaining a pathway database indicating a plurality of gene correlations between a plurality of genes and a plurality of biochemical pathways that influence a plurality of phenotypical health traits;
maintaining an activity database indicating a plurality of activity correlations between a plurality of human activities and a plurality of biochemical pathway effects;
receiving a DNA test result that includes a plurality of gene indicators that indicate a genetic makeup of an individual;
using the pathway database and the gene indicators to identify a plurality of influenced biochemical pathways of the individual;
using the activity database to identify an activity that affects at least one of the influenced biochemical pathways; and
recommending the activity to the individual to enhance expression of a gene that influences a phenotypical health trait that is one of the phenotypical health traits.

2. The method of claim 1 further including:
identifying an expressed phenotypical health trait of the individual, wherein the activity is recommended to enhance the expressed phenotypical health trait.

3. The method of claim 2 wherein the expressed phenotypical health trait is selected based on an online questionnaire.

4. The method of claim 2 wherein the expressed phenotypical health trait is identified using a telepresence interview.

5. The method of claim 1 wherein the activity is eating a food that enhances the phenotypical health trait via the at least one of the influenced biochemical pathways.

6. The method of claim 1 wherein the activity is avoiding a food that promotes the phenotypical health trait via the at least one of the influenced biochemical pathways.

7. The method of claim 1 wherein the activity is taking a dietary supplement that suppresses the phenotypical health trait via the at least one of the influenced biochemical pathways.

8. The method of claim 1 wherein the gene is one of a plurality of genes that govern a biochemical pathway associated with the phenotypical health trait.

9. The method of claim 1 wherein a second gene influences the phenotypical health trait and influences a second phenotypical health trait, wherein the activity does not enhance expression of the second gene.

10. The method of claim 1 further including:
identifying a second gene that influences the phenotypical health trait; and
determining that the second gene also influences a second phenotypical health trait, wherein
the activity is selected instead of a second activity that enhances expression of the gene and the second gene.

11. A system comprising:
a pathway database that indicates a plurality of gene correlations between a plurality of genes and a plurality of biochemical pathways that influence a plurality of phenotypical health traits;
an activity database indicating a plurality of activity correlations between a plurality of human activities and a plurality of biochemical pathway effects;
a memory; and
a processor communicatively coupled to the memory, wherein the processor is configured to:
receive a DNA test result that includes a plurality of gene indicators that indicate a genetic makeup an individual;
use the pathway database and the gene indicators to identify a plurality of influenced biochemical pathways of the individual;
use the activity database to identify an activity that affects at least one of the influenced biochemical pathways; and
produce a recommendation for the individual to perform the activity to enhance expression of a gene that influences a phenotypical health trait that is one of the phenotypical health traits.

12. The system of claim 11 wherein an expressed phenotypical health trait of the individual is determined using an online questionnaire.

13. The system of claim 12 wherein the activity is recommended to enhance the expressed phenotypical health trait.

14. The system of claim 11 wherein the phenotypical health trait is suppressed by the activity via the at least one of the influenced biochemical pathways.

15. The system of claim 11 wherein:
a second gene is identified that influences the phenotypical health trait and influences a second phenotypical health trait; and
the activity does not enhance expression of the second gene.

16. The system of claim 11 wherein:
a second gene influences the phenotypical health trait;
the second gene also influences a second phenotypical health trait; and
the activity is selected instead of a second activity because expression of the second gene is enhanced by the second activity.

17. A system comprising:
a means for identifying a plurality of influenced biochemical pathways of an individual based on a means for indicating a genetic makeup of the individual;
a means for identifying an activity that affects at least one of the influenced biochemical pathways; and
a means for recommending the activity for the individual to enhance expression of a gene that influences a phenotypical health trait that is one of the phenotypical health traits.

18. The system of claim 17 further including:
a means for identifying an expressed phenotypical health trait of the individual, wherein the activity is recommended to enhance the expressed phenotypical health trait.

19. The system of claim 17 wherein the gene is one of a plurality of genes that govern a biochemical pathway associated with the phenotypical health trait.

20. The system of claim 17 further including:
a means for identifying a second gene that influences the phenotypical health trait;
a means for determining that the second gene also influences a second phenotypical health trait; and
a means for not recommending a second activity wherein expression of the gene and of the second gene is enhanced by the second activity.
